(19) United States
(12) Patent Application Publication
(10) Pub. No.: US 2025/0091921 A1
(43) Pub. Date: Mar. 20, 2025

(54) DETECTION SYSTEM OF BIODEGRADATION REACTOR AND METHOD THEREOF

(71) Applicant: WUYI University, Jiangmen (CN)

(72) Inventors: Changyu LIU, Jiangmen (CN); Haowei ZHOU, Jiangmen (CN); Song ZHAO, Jiangmen (CN); Mengchen ZHANG, Jiangmen (CN); Xiaolong XU, Jiangmen (CN); Jianbo JIA, Jiangmen (CN)

(21) Appl. No.: 18/577,882

(22) PCT Filed: Nov. 6, 2023
(86) PCT No.: PCT/CN2023/129931

§ 371 Date: Jan. 9, 2024

(30) Foreign Application Priority Data
Aug. 17, 2023 (CN) 202311035930.6

(51) Int. Cl. C02F 3/10; C02F 103/02

(52) U.S. Cl. CPC C02F 3/108; C02F 2103/02; C02F 2209/06; C02F 2209/08; C02F 2209/14

(57) ABSTRACT

The present disclosure discloses a detection system of a biodegradation reactor and a method thereof. The detection system of the biodegradation reactor comprises a plug-flow microbial membrane reactor, a power system, a detection system and a flow system for storing and conveying a fluid. The detection analysis of a target pollutant is realized by detecting the consumption of a reactant or the generation of a product in a biodegradation reaction process, which can be applied to the detection of a water environmental pollutant. According to the present disclosure, various microorganisms with specific biodegradation functions in a water body are used to construct the biodegradation reactor, and a specific biodegradation function of the microbial membrane reactor is realized by the acclimation of a specific pollutant, so that tedious processes such as microbial separation and purification and culture in conventional methods are avoided, and the method is simple to operate.

### CROSS-REFERENCE TO RELATED APPLICATIONS

The present application is a national phase entry under 35 USC § 371 of International Application PCT/CN2023/129931 filed Nov. 6, 2023, which claims the benefit of and priority to Chinese Patent Application No. 202311035930.6, filed Aug. 17, 2023, the entire disclosures of which are incorporated herein by reference.

### TECHNICAL FIELD

The present disclosure belongs to the technical field of water quality monitoring, and particularly relates to a detection system of a biodegradation reactor and a method thereof.

### BACKGROUND

There are abundant microbial resources in nature, and the long-term induction of many factors in environment has acclimated environmental microorganisms capable of biodegrading various environmental pollutants, so as to maintain the self-purification and material circulation of ecosystem. A special degradation capability of microorganisms has been widely used in the field of water environmental pollutant treatment and detection. A water quality detection technology developed by using the microorganisms is mainly based on a microbial sensor method constructed by an immobilized microbial membrane and a microbial fuel cell method.

A microbial sensor is usually formed by coating a membrane on a surface of a dissolved oxygen (DO) sensing probe, wherein the membrane is formed by embedding a single bacterium or multiple bacteria purified and cultured through an immobilized material. The microbial sensor is usually used for determining a biochemical oxygen demand (BOD) of water quality. In the microbial sensor method, microorganisms need to be separated, purified, cultured and immobilized, so that the method is complicated to operate, and the immobilized and cultured microorganisms need to maintain the biological activity in a phosphate buffer solution. The immobilized microorganisms have a single population, a small quantity and a limited biodegradation capability. In a practical application process, environmental water samples are complex in composition, artificially screened strains have poor adaptability, and embedding materials are prone to blockage and swelling to cause microbial inactivation.

In an activated sludge aeration method, an activated sludge is taken as a microbial reaction unit, organic pollutants in the water samples are degraded by activated sludge aeration, and a BOD content in water is quantified by testing changes of chemical oxygen demand (COD) before and after biodegradation. In a practical application process, a process of decomposing the pollutants with the activated sludge is inevitably accompanied by microbial proliferation, which is easy to cause sludge bulking. Meanwhile, sludge proliferation may lead to a situation that an original standard curve is no longer applicable, and test results have poor stability.

A microbial fuel cell is an electrochemical system for converting a biochemical reaction into electrical energy, a microbial membrane is formed by enriching electrochemically active bacteria on an anode surface of the fuel cell, and chemical energy in an organic matter is converted into electrical energy by using electricity-generating microorganisms on the microbial membrane. The microbial fuel cell is often used for determining water quality parameters such as a BOD and a COD. Because the microbial fuel cell is an electrochemical system for converting a biochemical reaction into electrical energy, electrons generated by an organic matter of anaerobic metabolism of electroactive bacteria need to permeate through an electroactive bacterial membrane to reach an anode, and then are conveyed to a cathode through an external circuit to form a closed circuit. It is necessary to strictly control an anaerobic condition in an anode chamber in a running process. In an actual application, proliferation inevitably occurs in a process of metabolizing organic pollutants by electroactive bacteria, so that a thickness of the microbial membrane is increased, the efficiency of conveying the electrons to the anode is directly limited, the improvement of biodegradation efficiency of the electroactive bacterial membrane is accompanied by the gradual decrease of electron conveying efficiency, thus failing to achieve an expected result of an actual application effect. The complex types of organic pollutants in actual waste water and the conductivity and pH of solution may all affect the electricity production and microbial activity of the microbial fuel cell, resulting in an actual application effect not as good as expected.

At present, there are still some problems in the water quality detection technology based on the development of microorganisms, such as the complicated manual screening and culture of microorganisms, the poor stability of practical application of immobilization method and the low anti-interference capability of biological recognition element. Therefore, it is still necessary to find an efficient, accurate, stable and sensitive water quality detection method.

### SUMMARY

Aiming at the above defects of water quality detection in the prior art, the present disclosure will provide a construction method of a biodegradation reactor and a method for detecting multiple water quality parameters by the biodegradation reactor, the biodegradation reactor is constructed by using various microorganisms with specific biodegradation functions in a water body, a specific biodegradation function of a microbial membrane reactor is realized by the acclimation of a specific pollutant, and the detection analysis of a target pollutant is realized by detecting the consumption of a reactant or the generation of a product in a biodegradation reaction process.

In order to achieve the above objective, the following technical solution is provided specifically.

A plug-flow microbial membrane reactor comprises a plug-flow reactor and a microbial membrane, wherein the plug-flow reactor has a hollow tubular structure, and the microbial membrane is attached to an inner surface of the plug-flow reactor;
a construction method of the microbial membrane reactor comprises the following steps of:
(1) placing an environmental water sample and the plug-flow reactor in a constant-temperature water bath, and aerating the environmental water sample and then continuously conveying the environmental water sample to the plug-flow reactor through a pipeline, wherein the environmental water sample flows in the plug-flow reactor, and during a contact with the inner surface of the plug-flow reactor, microorganisms in the environmental water sample are attached to the inner surface of the plug-flow reactor to grow, so as to form the microbial membrane; and
(2) adding an acclimating agent into the environmental water sample, and then conveying the environmental water sample containing the acclimating agent to the plug-flow reactor for carrying out a primary acclimation and culture process of the microbial membrane, so as to obtain the microbial membrane reactor, wherein the acclimating agent comprises at least one of a biodegradable organic matter, an inorganic ammonium salt, inorganic carbonate and inorganic nitrite.

As a preferred embodiment of the present disclosure, the primary acclimation and culture process of the microbial membrane further comprises a monitoring method for the primary acclimation and culture process of the microbial membrane, which comprises the following steps of:
(2-1) conveying a blank water sample to the plug-flow reactor, and detecting a water sample flowing out through a detection system, so as to obtain an initial signal;
(2-2) adding a standard solution of a pollutant with a known concentration into the blank water sample, conveying the water sample containing the standard solution of the pollutant with the known concentration to the plug-flow reactor, and detecting a water sample flowing out through a detection system, so as to obtain a water sample signal, wherein a difference between the water sample signal and the initial signal is taken as a response signal of the water sample; and
(2-3) judging a growth status of the microbial membrane by a change of the response signal, and obtaining the microbial membrane reactor when it is measured that the response signal of the water sample does not change any more.

As a preferred embodiment of the present disclosure, in the standard solution of the pollutant in the step (2-2), when the biodegradation reactor is used for detecting a biochemical oxygen demand of water quality, the standard solution of the pollutant is a solution of the biodegradable organic matter, and a five-day biochemical oxygen demand (BOD₅) concentration of the biodegradable organic matter is 2 mg/L to 500 mg/L, when the biodegradation reactor is used for detecting ammonia nitrogen of water quality, the standard solution of the pollutant is a solution of the inorganic ammonium salt, an ammonia nitrogen concentration of the inorganic ammonium salt is 0.5 mg/L to 10 mg/L according to N, and when the biodegradation reactor is used for synchronously detecting the biochemical oxygen demand and the ammonia nitrogen of water quality, the standard solution of the pollutant is a mixed solution of the above two solutions.

As a preferred embodiment of the present disclosure, the construction method further comprises carrying out a secondary acclimation and culture process of the microbial membrane for the microbial membrane reactor, which specifically comprises the following steps of: adding an acclimating agent into a water sample in a constructed microbial membrane reactor, and then conveying the water sample containing the acclimating agent to the plug-flow reactor for carrying out the secondary acclimation and culture process of the microbial membrane, so as to obtain the microbial membrane reactor, wherein the acclimating agent used in the secondary acclimation and culture process of the microbial membrane is different from the acclimating agent used in the primary acclimation and culture process of the microbial membrane. A microbial population structure on the microbial membrane reactor may be dynamically adjusted by the acclimation with different acclimating agents, thus realizing different biodegradation capabilities of the plug-flow microbial membrane reactor. The microbial membrane reactor obtained by culture through the above steps is acclimated and cultured again with another acclimating agent, so as to obtain a microbial membrane reactor with another biodegradation function.

The present disclosure provides a microbial membrane reactor in a biodegradation reactor constructed by colonizing environmental microorganisms on a surface of a substrate, a specific biological reaction in the reactor is realized by the directional acclimation of a specific pollutant, and the detection analysis of a target pollutant is realized by detecting the consumption of a reactant and the generation of a product (conservation of material conversion) in a biodegradation reaction process.

Specifically: firstly, the hollow tubular structure is taken as the substrate and connected to the flow system for the construction of the plug-flow reactor, the water sample is in graded contact with the substrate in a continuous flowing process in the reactor, and the microbial membrane can only grow in a laminated manner in a confined space; and the adsorption, laminated growth and membrane formation of the environmental microorganisms in the water sample on the surface of the substrate are realized by using the surface adhesion of microorganisms, and a biodegradation reactor with a broad spectrum and a large quantity of microbial populations is constructed. Then, when the microorganisms are obviously adsorbed on a surface of the reactor, the specific pollutant is added into the water sample to acclimate and culture the microbial membrane, a signal detection unit is connected to a tail end of the plug-flow reactor, a standard solution of a pollutant to be detected with a certain concentration is introduced into the plug-flow reactor, whether the biochemical degradation reaction is carried out is judged according to a response situation of the signal detection unit, a response signal of the microbial membrane to a standard solution with the same concentration is tracked regularly to judge the growth status of the microbial membrane, and when the standard solution with the same concentration is repeatedly measured for many times and the response signal does not change any more, the culture of the microbial membrane is completed. In this process, the microorganisms on the microbial membrane are also acclimated by continuous and frequent standard sample tests, and dominant microbial strains under this condition are gradually screened out in a continuous acclimation process, so that the pollutant is efficiently degraded to accurately quantify a content of the pollutant.

The growth status of the microbial membrane is judged by the change of the response signal, and the microbial membrane reactor is obtained when it is measured that the response signal of the water sample does not change any more; and when it is measured that the response signal of the water sample still changes, a process of sequentially carrying out the steps (2), (2-1) and (2-2) is repeated for several times, and an interval between the step (2) and the steps (2-1) and (2-2) is 1 hour to 4 hours each time.

As a preferred embodiment of the present disclosure, repeating the process of sequentially carrying out the steps (2), (2-1) and (2-2) for several times specifically comprises: the interval each time is 4 hours, when response signals measured for two consecutive times are less than 0.2 mg/L, the interval each time is changed into 1 hour, and when response signals measured for three consecutive times do not change any more, the microbial membrane reactor is obtained.

According to the present disclosure, various microorganisms with specific biodegradation functions in a water body are used to construct the biodegradation reactor, and a specific biodegradation function of the microorganisms is realized by the directional acclimation of a specific environmental pollutant, so that tedious processes such as microbial separation and purification and culture in conventional methods are avoided, and the method is simple to operate; biological reaction units for degrading different pollutants in a targeted manner may be acclimated by using different environmental pollutants, so that the method has a wide application range; a composition of the microbial population on the microbial membrane reactor may be adjusted by an acclimation technology, so that the alternating/simultaneous on-line monitoring of different environmental pollutants is realized; the naturally acclimated microbial population has excellent environmental adaptability and strong anti-interference capability, so that an actual water sample may be accurately measured without pretreatment; and the construction of the plug-flow reactor realizes the graded contact between the pollutant and the microbial reaction unit, so that high biodegradation efficiency is achieved, and the analysis method is high in sensitivity.

As a preferred embodiment of the present disclosure, the biodegradable organic matter comprises at least one of biodegradable saccharide, amino acid, aliphatic hydrocarbon, an organic acid and an alcohol organic matter; the inorganic ammonium salt comprises an inorganic matter containing ammonium ions; the inorganic carbonate comprises an inorganic matter containing carbonate ions; and the inorganic nitrite comprises an inorganic matter containing nitrite ions.

As a preferred embodiment of the present disclosure, a five-day biochemical oxygen demand (BOD₅) concentration of the biodegradable organic matter is 2 mg/L to 500 mg/L, an ammonia nitrogen concentration of the inorganic ammonium salt is 0.5 mg/L to 50 mg/L according to N, a mass concentration of the inorganic carbonate is 0.004 g/L to 0.4 g/L, and a nitrite nitrogen concentration of the inorganic nitrite is 0.05 mg/L to 5 mg/L according to N.

As a preferred embodiment of the present disclosure, the detection system comprises at least one of a dissolved oxygen analysis system, a total organic carbon (TOC) analysis system, a chemical oxygen demand (COD) analysis system and a pH analysis system.

As a preferred embodiment of the present disclosure, the blank water sample comprises a tap water sample or an unpolluted water sample.

As a preferred embodiment of the present disclosure, the environmental water sample comprises at least one of a natural water body and waste water.

As a preferred embodiment of the present disclosure, the water sample comprises at least one of river water, an effluent from a primary sedimentation tank of a domestic waste water treatment plant, pond water, and lake and reservoir water.

As a preferred embodiment of the present disclosure, the plug-flow reactor is made of a material comprising at least one of polyurethane, polylactic acid and glass.

As a further preferred embodiment of the present disclosure, the plug-flow reactor is a hollow multi-channel cylindrical pipeline with a length of 75 cm and an outer diameter of 2.4 cm, wherein an inner diameter of each channel is 1 mm to 3 mm, or a spiral tubular pipeline with a wall thickness of 0.5 mm, an inner diameter of 1 mm to 4 mm and a total length of 100 cm to 200 cm.

Biocompatible materials such as polyurethane and polylactic acid are taken as the substrate, important parameters such as a cross-sectional shape of a structure cavity of a reactor, a channel size, a hydraulic flow direction and a reactor volume are designed, and the reactor is designed by heat setting and 3D printing, and connected to the flow system to construct the plug-flow reactor.

As a preferred embodiment of the present disclosure, a temperature of the constant-temperature water bath is 25° C. to 37° C.

As a preferred embodiment of the present disclosure, a flow rate of the environmental water sample is 0.5 mL/min to 5 mL/min.

The water sample is aerated and then continuously introduced into the plug-flow reactor, the water sample flows in the plug-flow reactor at a constant speed, and during the contact with the surface of the substrate, the microorganisms are attached to the surface of the substrate to grow, so as to gradually form the microbial membrane. The constant-speed graded contact allows the microorganisms to be evenly attached in the reactor.

A detection system of a biodegradation reactor comprises the plug-flow microbial membrane reactor, a power system, a detection system and a flow system for storing and conveying a fluid; and
one side of the plug-flow microbial membrane reactor is connected with a pipeline and the power system to form a closed system, a fluid in the flow system is conveyed to the plug-flow microbial membrane reactor through the pipeline and the power system, the fluid passes through the plug-flow microbial membrane reactor and then flows out from another side, and a fluid flowing out is detected through the detection system.

As a preferred embodiment of the present disclosure, the power system comprises a peristaltic pump.

The power system provides power for the fluid, and is configured for controlling the fluid in the flow system to flow in and out; and the plug-flow microbial membrane reactor is configured for the biodegradation of the environmental pollutant, wherein, in an acclimation process of the microbial membrane, a plug-flow microbial membrane reactor with a specific biodegradation function is obtained after the acclimation with the acclimating agent, the pollutant passing through the reactor is degraded through the specific degradation function, and in combination with a detection function of the detection system on an effluent, the purpose of detecting the concentration of the pollutant is achieved through degradation-detection.

A detection method of a detection system of a biodegradation reactor comprises the following steps of:
(1) taking a blank water sample as a fluid, and detecting the blank water sample by using the detection system of the biodegradation reactor, so as to obtain an initial signal; and
(2) detecting a signal of a water sample to be detected through the detection system of the biodegradation reactor, so as to obtain the signal of the water sample to be detected, wherein a difference between the signal of the water sample to be detected and the initial signal is taken as a response signal of the water sample to be detected.

As a preferred embodiment of the present disclosure, the detection method of the detection system of the biodegradation reactor further comprises the following steps of:
(1) drawing a standard curve: taking standard samples containing pollutants with different concentrations as standard solutions, detecting the standard solutions by using the detection system of the biodegradation reactor, so as to obtain response signals of different standard solutions, and taking the concentrations of the standard solutions as abscissas and taking the response signals of the standard solutions as ordinates to draw the standard curve; and
(2) detecting the water sample to be detected: detecting the water sample to be detected by using the detection system of the biodegradation reactor, so as to obtain the response signal of the water sample to be detected, and calculating a detection result of the water sample to be detected through the standard curve.

When used for detecting a biochemical oxygen demand (BOD) of water quality, the acclimating agent is a biodegradable organic matter, and the detection system is a dissolved oxygen analysis system, a total organic carbon (TOC) analysis system or a chemical oxygen demand (COD) analysis system.

When used for detecting an ammonia nitrogen concentration of water quality, the acclimating agent is an inorganic ammonium salt, inorganic carbonate and/or inorganic nitrite, and the detection system is a pH analysis system or a dissolved oxygen analysis system.

When used for synchronously detecting the BOD and the ammonia nitrogen of water quality, the acclimating agent is a biodegradable organic matter, an inorganic ammonium salt, inorganic carbonate and/or inorganic nitrite, and the detection system is a dissolved oxygen analysis system.

When the water quality is detected by using the biodegradation reactor, the unpolluted water sample is used first, such as a tap water background solution, placed in a constant-temperature condition, aerated and then continuously introduced into the reactor, an effluent of the reactor is subjected to signal detection, and at the moment, the signal response is a blank signal of the microbial membrane; when the sample to be detected flows through the reactor, the biodegradation reaction occurs, which is accompanied by material conversion, a difference between the response signal and the blank signal measured by the signal detection unit is a degree of biochemical degradation of the pollutant by the microbial membrane in the reactor, and this signal has a corresponding relationship with the concentration of the pollutant.

The standard curve is drawn by testing response signals of several standard samples with known concentrations, and the concentration of the pollutant of the water sample to be detected is calculated according to a corresponding relationship of the standard curve. After ending a single test, an unpolluted water sample is continuously introduced into the reactor to discharge a residual sample before the next measurement.

In use of the biodegradation reactor in the detection of water quality, the alternating/synchronous monitoring of the water quality parameters comprising the BOD and the ammonia nitrogen is realized by using the specific biodegradation function.

Compared with the prior art, the present disclosure has the following beneficial effects.

(1) According to the biodegradation reactor of the present disclosure, the plug-flow reactor is constructed in the hollow tubular structure, so that the environmental microorganisms are evenly attached to form the membrane during the contact between the water sample and the substrate, the microbial membrane reactor may be constructed without using an embedding material and an expensive carrier, the microbial membrane can only grow in a laminated manner in a confined space, under the limitation of mass transfer efficiency of a substance in the microbial membrane, only the microbial membrane on a surface layer participates in the biodegradation reaction, and the biodegradation efficiency is not affected by the proliferation of the microbial membrane, thus realizing the long-term stability of the microbial membrane.

(2) In the biodegradation reactor of the present disclosure, the environmental microorganisms contained in the natural water body itself are products of natural acclimation and survival competition, and usually belong to the microbial population most suitable for degrading the pollutant contained in the water body. The environmental microorganisms are colonized on the surface of the substrate to construct the microbial membrane, and meanwhile, the specific biodegradation capability of the microbial membrane is realized by the further acclimation of the environmental pollutant, so that the microbial membrane is used for determining the pollutant in the water sample, which can best reflect an intrinsic biochemical reaction process of the microbial population in this water body, and meanwhile, the naturally acclimated microbial population has excellent environmental adaptability, a strong anti-interference capability, and efficient and stable biodegradation, and a water quality detection and analysis method developed on this basis has high sensitivity.

(3) In the biodegradation reactor of the present disclosure, the environmental microorganisms are acclimated by using the environmental pollutant to realize biodegradation simplification, thus avoiding tedious processes of microbial separation and purification and culture, and microbial activity is maintained by using the tap water without using a buffer system in an application process.

(4) The biodegradation reactor of the present disclosure is taken from the environment and used in the environment, and is simple to operate, free from reagent consumption, and green and environment-friendly, and meanwhile, the water quality detection process also realizes the partial degradation of the pollutant.

(5) According to the present disclosure, the composition of the microbial population on the microbial membrane reactor may be adjusted by acclimation to realize the alternating/simultaneous on-line monitoring of different environmental pollutants, so that the BOD or the ammonia nitrogen may be monitored separately or synchronously in a targeted manner.

(6) A microbial population structure on the microbial membrane reactor may be dynamically regulated by the acclimation with different acclimating agents, thus realizing different biodegradation capabilities of the plug-flow microbial membrane reactor, for example, a microbial membrane reactor with another biodegradation function may be obtained by changing the acclimating agent to acclimate and culture the microorganisms again, and in the present disclosure, the detection signal and the microbial membrane reactor may be changed to realize the detection of different pollutants.

### DESCRIPTION OF THE DRAWINGS

FIG. 1 is a structural diagram of an environmental pollutant biodegradation reactor according to an example of the present disclosure.

FIG. 2A, FIG. 2B and FIG. 2C are schematic diagrams of a multi-channel plug-flow reactor according to an example of the present disclosure, wherein FIG. 2A is a front view of the reactor, FIG. 2B is a schematic diagram of a hollow multi-channel cylindrical pipeline of the reactor, and FIG. 2C is a cross-sectional view of the reactor.

FIG. 3 is a standard curve diagram of BOD according to Example 1.

FIG. 4 is a relationship curve diagram of ATOC and a BOD according to Example 2.

FIG. 5 is a standard curve diagram of ammonia nitrogen (NH⁺₄—N) according to Example 3.

FIG. 6 is a standard curve diagram of BOD according to Example 4.

FIG. 7 is a standard curve diagram of ammonia nitrogen (NH⁺₄—N) according to E 4.

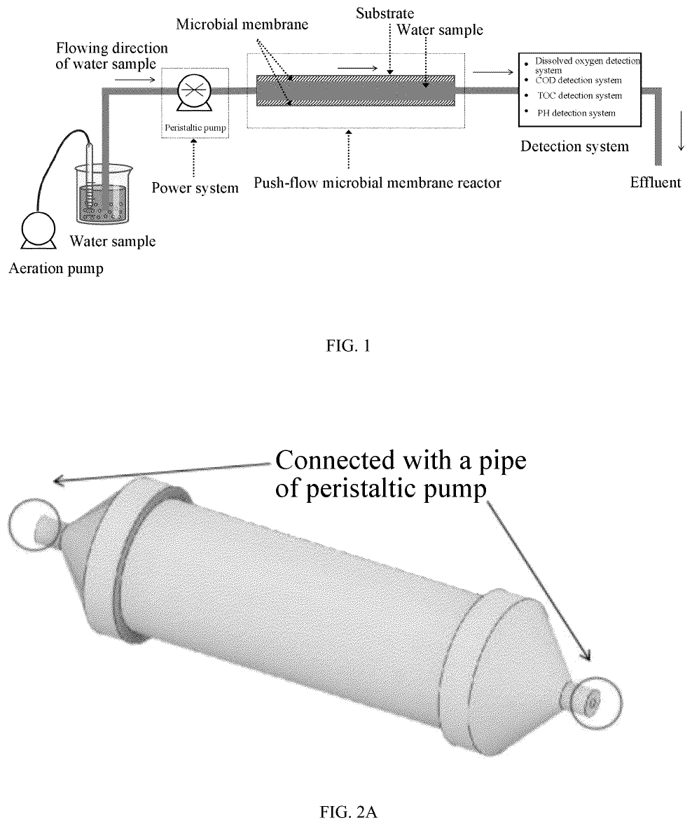

### DETAILED DESCRIPTION

In order to better explain the objectives, technical solutions and advantages of the present disclosure, the present disclosure will be further described hereinafter with reference to specific examples.

### Example 1

A biodegradation reactor of this example used a dissolved oxygen electrode as a signal detection unit, and was used for the detection of a biochemical oxygen demand (BOD) of water quality.

A construction method of the biodegradation reactor comprised the following steps.

### (1) Design of Push-Flow Reactor

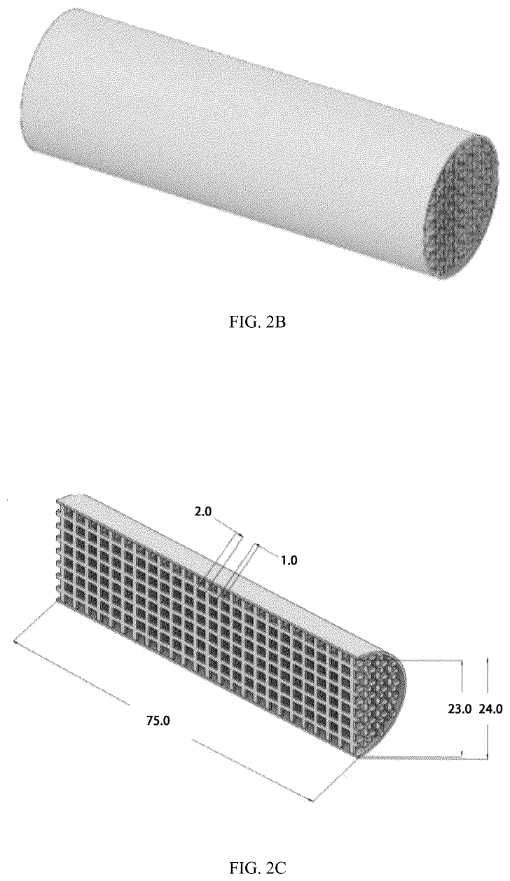

A hollow multi-channel cylindrical pipeline with a length of 7.5 cm and an outer diameter of 2.4 cm made of polylactic acid (PLA) was prepared by a 3D printing technology, as shown in FIG. 2A, FIG. 2B and FIG. 2C (the multi-channel design was intended to provide more adsorption surface area for microorganisms), wherein each channel had an inner diameter of 2 mm, and was connected to a power system through a hose.

### (2) Culture of Microbial Membrane Reactor

(2-1) Microbial adsorption: the plug-flow reactor was placed in a constant-temperature water bath at 37° C., and a water sample (river water sample) collected from river was subjected to air saturation through an aeration pump, and also placed in a constant-temperature water bath at 37° C. Under the driving of a peristaltic pump, the water sample was continuously conveyed to the plug-flow reactor in a single direction at a flow rate of 3 mL/min and then discharged. Meanwhile, an inlet water sample was continuously supplemented, the water sample was in graded contact with a substrate in this process, and environmental microorganisms in the water sample were gradually adsorbed and colonized on a surface of the substrate.

(2-2) Acclimation of microbial membrane: when obvious microbial micelles visible to naked eyes were adsorbed on an inner surface of the reactor, an acclimating solution was added into the water sample until a BOD concentration was 100 mg/L, and a resulting water sample was continuously introduced into the plug-flow reactor to acclimate and culture the microbial membrane. The acclimating solution was a mixed solution composed of a glucose solution, a glutamic acid solution, a galactose solution, a sucrose solution, a benzoic acid solution, a malic acid solution and an asparagine solution with a BOD concentration of 1,000 mg/L respectively in an equal ratio, and the acclimating solution was added into the water sample at a ratio of 1:10 during acclimation.

(2-3) Detection and monitoring of acclimation process of microbial membrane: the dissolved oxygen electrode was connected to a tail end of the plug-flow reactor as the signal detection unit, tap water was taken and added into a water sample cup, and placed in a constant-temperature water bath at 37° C. and then subjected to air saturation, the tap water was conveyed to the above plug-flow reactor at a flow rate of 3 mL/min by the peristaltic pump, the dissolved oxygen electrode was used as a signal detection system to detect an oxygen content when the water sample reached the oxygen electrode, and when a dissolved oxygen signal reached a steady state, the signal was recorded as DO_{b}. A glucose-glutamic acid (GGA) standard solution with a BOD concentration of 10 mg/L was introduced, and when the GGA solution entered the reactor, microorganisms on the microbial membrane degraded an organic matter during the contact with the organic matter in the solution. Meanwhile, dissolved oxygen in water was consumed in this process, a dissolved oxygen content was reduced when the water sample reached a surface of the oxygen electrode, and when the signal reached the steady state again, the signal was recorded as DOₛ. A difference ADO between the DO_{b} and the DOₛ was the signal response of the reactor to the GGA solution. This process of the step (2-3) was also a test flow when the biodegradation reactor was used for detecting a pollutant of water quality.

(2-4) The test was carried out with the GGA standard solution with the BOD concentration of 10 mg/L every 4 hours, and comprised specific steps as follows: the above steps of culturing the microbial membrane and testing the standard sample were repeated (that was, the steps 2-2 and 2-3 were repeated) until the difference ADO between signal response values measured for 2 consecutive times was less than 0.2 mg/L, and a test interval of the standard sample was changed to once every hour. When signals measured for 3 consecutive times did not change any more, it was indicated that a stable microbial membrane was formed on an inner wall of the reactor, thus judging that the culture of the microbial membrane reactor formed by colonizing the environmental microorganisms was completed.

### (3) Drawing of Standard Curve

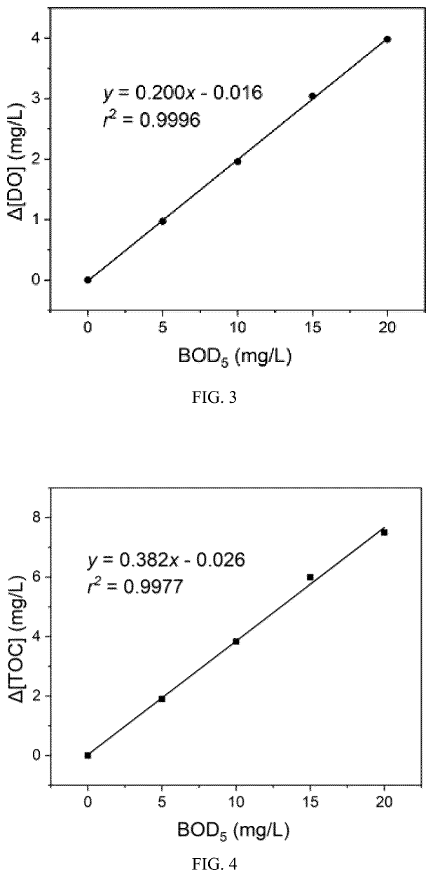

With reference to the above step (2-3), GGA standard solutions with BOD concentrations of 0.0 mg/L, 5.0 mg/L, 10.0 mg/L, 15.0 mg/L and 20.0 mg/L were respectively introduced into the above microbial membrane reactor in sequence to detect the GGA standard solutions, and a steady-state signal difference ADO of the oxygen electrode was recorded when the tap water and the standard solution passed through the microbial membrane and then reached the oxygen electrode. Results were shown in Table 1. The standard curve was drawn by taking the BOD concentrations as abscissas and the corresponding dissolved oxygen signal differences ΔDO as ordinates. Results showed that the standard curve had an equation of y=0.200x−0.016, a linear range of 0 mg/L to 20 mg/L, and a correlation coefficient of r²=0.9996, as shown in FIG. 3.

TABLE 1
Data of standard curve of BOD

| | | | | | |
|---|---|---|---|---|---|
| BOD concentration mg/L | 0.0 | 5.0 | 10.0 | 15.0 | 20.0 |
| Dissolved oxygen difference ΔDO mg/L | 0.0 | 0.97 | 1.96 | 3.04 | 3.98 |

| | |
|---|---|
| Formula of standard curve | y = 0.200x − 0.016 |

### (4) Test of River Water Sample

Tap water and river water samples were taken and added into beakers respectively, placed in a constant-temperature water bath at 37° C., and then subjected to air saturation at 3.5 L/min, and the tap water was continuously conveyed to the reactor at a flow rate of 3 mL/min by a peristaltic pump and then flowed through the oxygen electrode. When an output signal of the oxygen electrode reached a steady state, the dissolved oxygen signal was recorded, and then the river water sample was continuously introduced into the reactor. When the solution passed through the microbial membrane and reached the dissolved oxygen electrode, the electrode detected that the output signal was gradually decreased and then reached a new steady state, a dissolved oxygen change value was calculated in this process, and a BOD value of the water sample was calculated according to the standard curve.

### (5) Test of River Water Sample by National Standard Method (to Obtain True BOD Value of Water Sample)

The river water sample in the step (4) was subjected to biochemical culture for 5 days by a national standard dilution and inoculation method (HJ 505-2009), so as to obtain a BOD value of the river water sample.

### (6) Test Results

Test results of the water sample in the steps (4) and (5) were shown in Table 2.

TABLE 2
Comparison of test results of BOD value of river water sample

| Sample | River water |
|---|---|
| Detection result of method in this example mg/L | 3.4 |
| Test result of national standard method mg/L | 3.3 |
| Relative error % | 3.0% |

### Example 2

A biodegradation reactor in this example rapidly determined a BOD value of a water sample by testing a difference of total organic carbon (TOC) before and after a biodegradation reaction.

A construction method of the biodegradation reactor referred to Example 1, which was different from the construction method in Example 1 in that: the plug-flow reactor was designed by using a polyurethane pipeline with an inner diameter of 3 mm and a length of 110 cm, and the microbial membrane grew on an inner wall of the pipeline; an environmental water sample used for culturing the microbial membrane was an effluent from a primary sedimentation tank of a domestic waste water treatment plant; an acclimating agent used for acclimating the microbial membrane was a glucose-glutamic acid (GGA) solution; and a flow rate of the sample in the plug-flow reactor was 1.5 mL/min. When a BOD was detected, the detection system was a TOC analyzer. The water sample was detected by the TOC analyzer first, so as to obtain an initial TOC value of the water sample, then the water sample was conveyed to the plug-flow reactor, and an effluent from the reactor was collected and TOC was measured, so as to obtain a TOC value of the water sample after passing through the biodegradation reaction. The BOD of water quality was determined by using a corresponding relationship between a TOC difference before and after the biodegradation reaction and the BOD value, and a relationship curve diagram of the ATOC and the BOD was shown in FIG. 4. Test results of the domestic waste water sample were shown in Table 3.

TABLE 3
Comparison of test results of BOD value
of domestic waste water sample

| Sample | Domestic waste water |
|---|---|
| Detection result of method in this example mg/L | 11.5 |
| Test result of national standard method mg/L | 12.3 |
| Relative error % | 6.5% |

### Example 3

A biodegradation reactor of this example used a dissolved oxygen electrode as a signal detection unit, and was used for the detection of ammonia nitrogen of water quality.

A construction method of the biodegradation reactor comprised the following steps.

### (1) Design of Push-Flow Reactor

A glass material was used as a raw material to be fired into a spiral tubular pipeline with a wall thickness of 0.5 mm, an inner diameter of 2.4 mm and a total length of 150 cm, washed and then introduced with an HF solution with a concentration of 10% to etch a glass inner wall for surface roughening, and an etching solution was discharged 15 minutes later, washed with pure water to remove the residual solution, and then dried. The spiral tubular pipeline was connected to a power system through a silica gel hose.

### (2) Culture of Microbial Membrane Reactor

(2-1) Microbial adsorption: the plug-flow reactor was placed in a constant-temperature water bath at 30° C., and a water sample (pond water) collected from a pond was subjected to air saturation through an aeration pump, and also placed in a constant-temperature water bath at 30° C. Under the driving of a peristaltic pump, the water sample was continuously conveyed to the plug-flow reactor in a single direction at a flow rate of 3.5 mL/min and then discharged. Meanwhile, an inlet water sample was continuously supplemented, the water sample was in graded contact with a substrate in this process, and environmental microorganisms in the water sample were gradually adsorbed and colonized on a surface of the substrate.

(2-2) Acclimation of microbial membrane: when obvious microbial micelles visible to naked eyes were adsorbed on a surface of the reactor, an acclimating solution was added into the water sample to acclimate and culture the microbial membrane. The acclimating solution was a mixed solution composed of an ammonium chloride solution with a mass concentration of 3.819 g/L, a sodium bicarbonate solution with a mass concentration of 7.638 g/L and a sodium nitrite solution with a mass concentration of 0.492 g/L, and the acclimating solution was added into the water sample at a ratio of 1:50 during acclimation. After long-term acclimation with the acclimating solution, nitrifying bacteria on the microbial membrane gradually proliferated, and heterotrophic microorganisms were gradually inactivated due to long-term lack of nutrient sources such as an organic matter.

(2-3) Monitoring of acclimation process of microbial membrane: the dissolved oxygen electrode was connected to a tail end of the plug-flow reactor as the signal detection unit, tap water was taken and added into a water sample cup, placed in a constant-temperature water bath at 30° C., and subjected to air saturation, the tap water was conveyed to the plug-flow reactor at a flow rate of 3.5 mL/min by the peristaltic pump, the dissolved oxygen electrode was used as a signal detection system to detect an oxygen content when the water sample reached the oxygen electrode, and when a dissolved oxygen signal reached a steady state, the signal was recorded as DO_{b}. Subsequently, an NH₄Cl solution with an ammonia nitrogen concentration of 1 mg/L was introduced, and when the NH₄Cl solution entered the reactor, microorganisms on the microbial membrane were subjected to a nitration reaction during the contact with the ammonia nitrogen in the solution. Meanwhile, dissolved oxygen in water was consumed in this process, a dissolved oxygen content was reduced when the water sample reached a surface of the oxygen electrode, and when the signal reached the steady state again, the signal was recorded as DOₛ. A difference ADO between the DO_{b} and the DOₛ was the signal response of the reactor to the NH₄Cl solution.

(2-4) The test was carried out with the NH₄Cl solution with the ammonia nitrogen concentration of 1 mg/L every 4 hours, and comprised specific steps as follows: the above steps of culturing the microbial membrane and testing the standard sample were repeated until the difference ADO between signal response values measured for 2 consecutive times was less than 0.2 mg/L, and a test interval of the standard sample was changed to once every hour. When signals measured for 3 consecutive times did not change any more, it was indicated that a stable microbial membrane was formed on an inner wall of the reactor, thus judging that the culture of the microbial membrane reactor formed by colonizing the environmental microorganisms was completed.

### (3) Drawing of Standard Curve

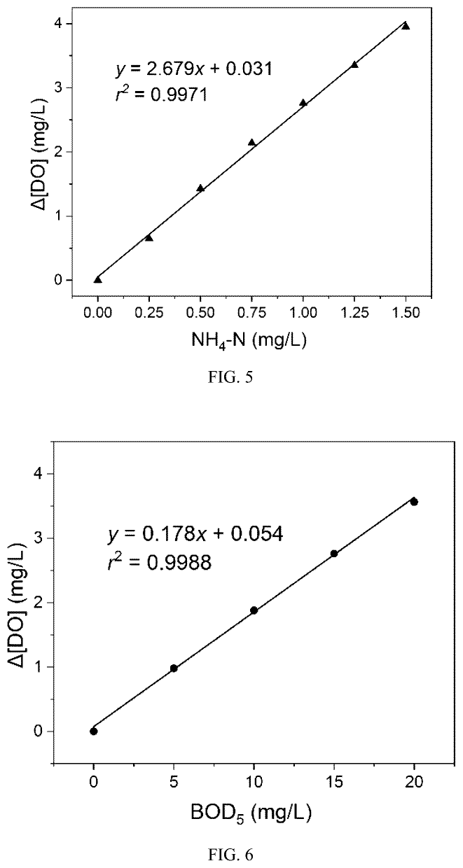

According to a flow of the step (2-3), NH⁺₄—N standard solutions with NH⁺₄—N concentrations of 0.0 mg/L, 0.25 mg/L, 0.5 mg/L, 0.75 mg/L, 1.0 mg/L, 1.25 mg/L and 1.5 mg/L were respectively introduced into the above microbial membrane reactor in sequence to carry out ADO detection on the NH⁺₄—N standard solutions, and a steady-state dissolved oxygen signal difference ADO was recorded when the tap water and the standard sample passed through the microbial membrane and then reached the oxygen electrode. Results were shown in Table 4. The standard curve was drawn by taking the NH⁺₄—N concentrations as abscissas and the corresponding dissolved oxygen differences ΔDO as ordinates. Results showed that an equation was y=2.679x+0.031, a linear range was 0 mg/L to 1.5 mg/L, and a correlation coefficient was r²=0.9971, as shown in FIG. 5.

TABLE 4
Data of standard curve of ammonia nitrogen

| | | | | | | | |
|---|---|---|---|---|---|---|---|
| Ammonia nitrogen | 0.00 | 0.25 | 0.50 | 0.75 | 1.00 | 1.25 | 1.50 |
| concentration mg/L | | | | | | | |
| Dissolved oxygen | 0.0 | 0.65 | 1.43 | 2.14 | 2.76 | 3.35 | 3.95 |
| difference ΔDO mg/L | | | | | | | |

| | |
|---|---|
| Formula of | y = 2.679x + 0.031 |
| standard curve | |

### (4) Test of Pond Water Sample

Tap water and pond water samples were taken and added into beakers respectively, placed in a constant-temperature water bath at 30° C., and then subjected to air saturation, and the tap water was continuously conveyed to the reactor at a flow rate of 3.5 mL/min by a peristaltic pump and then flowed through the oxygen electrode. When an output signal of the oxygen electrode reached a steady state, the dissolved oxygen signal was recorded, and then the pond water sample was continuously introduced into the reactor. When the solution passed through the microbial membrane and reached the dissolved oxygen electrode, the electrode detected that the output signal was gradually decreased and then reached a new steady state, a dissolved oxygen change value was calculated in this process, and an ammonia nitrogen value of the water sample was calculated according to the standard curve.

### (5) Test of Pond Water Sample by National Standard Method (to Obtain True Ammonia Nitrogen Value of Water Sample)

The water sample was tested by national standard salicylic acid spectrophotometry (HJ 536-2009) to obtain the ammonia nitrogen value.

### (6) Test Results

Test results of the water sample in the steps (4) and (5) were shown in Table 5.

TABLE 5
Comparison of test results of ammonia
nitrogen value of water sample

| Sample | Pond water |
|---|---|
| Detection result of method in this example mg/L | 0.68 |
| Test result of national standard method mg/L | 0.71 |
| Relative error % | −4.2% |

### Example 4

A biodegradation reactor of this example used a dissolved oxygen electrode as a signal detection unit, and was used for the synchronous determination of BOD and ammonia nitrogen of water quality.

The biodegradation reactor was obtained by subjecting to the microbial membrane reactor used in above Example 3 to secondary acclimation and culture, and comprised the following steps.

### (1) Secondary Acclimation and Culture of Microbial Membrane Reactor

(1-1) Acclimation of microbial membrane: the above plug-flow reactor was placed in a constant-temperature water bath at 35° C., a water sample collected from an effluent of a primary sedimentation tank of a domestic waste water treatment plant (waste water) was subjected to air saturation by an aeration pump, and also placed in a constant-temperature water bath at 35° C., and a mixed solution of GGA and NH₄Cl was added into the water sample until a BOD concentration was 100 mg/L and an ammonia nitrogen concentration was 10 mg/L, and a resulting water sample was introduced into the plug-flow reactor at a flow rate of 4 mL/min to acclimate and culture the microbial membrane, so that aerobic microorganisms and nitrifying bacteria in the water sample could obtain sufficient nutrient sources to maintain biodegradation activity and proliferate.

(1-2) Monitoring of microbial membrane acclimation process: the dissolved oxygen electrode was connected to a tail end of the plug-flow reactor as the signal detection unit, and the test was carried out with a GGA solution with a GGA concentration of 10 mg/L and an NH₄Cl solution with an ammonia nitrogen concentration of 1 mg/L respectively in sequence. Specific test steps were the same as those in above Example 1.

(1-3) The above steps of culturing the microbial membrane and testing the standard sample were repeated every 4 hours until the difference between signal response values measured for 2 consecutive times was less than 0.2 mg/L, and a test interval of the standard sample was changed to once every hour. When signals measured for 3 consecutive times did not change any more, it was indicated that a stable microbial membrane was formed on an inner wall of the reactor, thus judging that the culture of the microbial membrane reactor formed by colonizing the environmental microorganisms was completed.

### (2) Drawing of Standard Curve

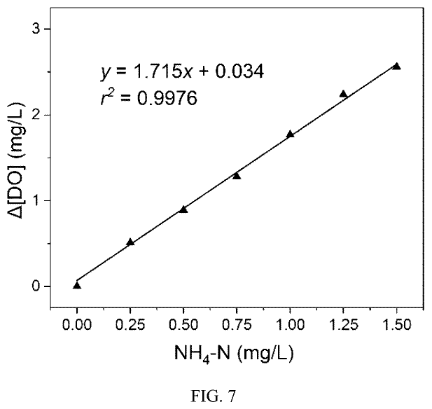

According to the above flow, a standard curve of BOD and a standard curve of ammonia nitrogen were drawn respectively. Results were shown in FIG. 6 and FIG. 7.

### (3) Test of Waste Water Sample

Tap water and waste water samples were taken and added into beakers respectively, placed in a constant-temperature water bath at 35° C., and then subjected to air saturation, and the tap water was continuously conveyed to the reactor at a flow rate of 4 mL/min and then flowed through the oxygen electrode. When an output signal of the oxygen electrode reached a steady state, the dissolved oxygen signal was recorded, and then the waste water sample was continuously introduced into the reactor. When the solution passed through the microbial membrane and reached the dissolved oxygen electrode, the electrode detected that the output signal was gradually decreased and then reached a new steady state, and a dissolved oxygen change value DOₜ was calculated in this process, which represented an amount of dissolved oxygen consumed in a process of biodegrading the organic matter and the ammonia nitrogen in the water sample by the microbial membrane. Subsequently, 0.02 mg/L acrylthiourea (ATU) solution was added into the waste water sample for the measurement in the above step, and a dissolved oxygen change value in this process was DO_{BOD}, which represented an amount of dissolved oxygen consumed in a process of biodegrading the organic matter in the water sample by the microbial membrane. It should be noted that an allylthiourea solution was usually used as a nitrification inhibitor to inhibit nitrification, which was a noncompetitive inhibitor, and could not inhibit microbial activity in the reactor under the concentration conditions used in this example. A BOD value of the water sample was calculated according to the DO_{BOD}, and an NH⁺₄—N value of the water sample was calculated according to DO_{NH+4-N}=DOₜ−DO_{BOD}.

(4) Test of Waste Water Sample by National Standard Method (to Obtain True BOD Value and NH⁺₄—N Value of Water Sample)

The waste water sample in the step (4) was subjected to biochemical culture for 5 days by a national standard BOD5 method, so as to obtain a BOD value of the waste water sample.

The NH⁺₄—N value of the waste water sample was obtained by a national standard method.

### (5) Test Results

Test results of the water sample in the steps (4) and (5) were shown in Table 6.

TABLE 6
Comparison of test results of BOD and
NH⁺₄—N values of waste water sample

| | Waste |
|---|---|
| Sample | water |
| BOD detection result of method in this example mg/L | 4.2 |
| BOD test result of national standard method mg/L | 4.1 |
| Relative error % | 2.4% |
| NH⁺₄—N detection result of method in this embodiment mg/L | 0.88 |
| NH⁺₄—N test result of national standard method mg/L | 0.90 |
| Relative error % | −2.2% |

It can be seen from Examples 1 to 4 that, according to the biodegradation reactor constructed by colonizing the environmental microorganisms on the surface of the substrate in the present disclosure, a specific biological reaction in the reactor is realized by the directional acclimation of a specific pollutant, and the detection analysis of a target pollutant is realized by detecting the consumption of a reactant and the generation of a product in a biodegradation reaction process. The method is simple to operate and wide in application range, the analysis method is high in sensitivity and free of reagent consumption, and the method is a green and environmentally-friendly method for the analysis and detection of the environmental pollutant.

Finally, it should be noted that the above examples are only used to illustrate the technical solutions of the present disclosure, and are not intended to limit the scope of protection of the present disclosure. Although the present disclosure is described in detail with reference to the preferred examples, those of ordinary skills in the art should understand that the technical solutions of the present disclosure can be modified or equivalently replaced without departing from the essence and scope of the technical solutions of the present disclosure.

1. A plug-flow microbial membrane reactor, comprising a plug-flow reactor and a microbial membrane, wherein the plug-flow reactor has a hollow tubular structure, and the microbial membrane is attached to an inner surface of the plug-flow reactor;
a construction method of the microbial membrane reactor comprises the following steps of:
(1) placing an environmental water sample and the plug-flow reactor in a constant-temperature water bath, and aerating the environmental water sample and then continuously conveying the environmental water sample to the plug-flow reactor through a pipeline, wherein the environmental water sample flows in the plug-flow reactor, and during a contact with the inner surface of the plug-flow reactor, microorganisms in the environmental water sample are attached to the inner surface of the plug-flow reactor to grow, so as to form the microbial membrane; and
(2) adding an acclimating agent into the environmental water sample, and then conveying the water sample containing the acclimating agent to the plug-flow reactor for carrying out a primary acclimation and culture process of the microbial membrane, so as to obtain the microbial membrane reactor, wherein the acclimating agent comprises at least one of a biodegradable organic matter, an inorganic ammonium salt, inorganic carbonate and inorganic nitrite.

2. The plug-flow microbial membrane reactor according to claim 1, wherein the primary acclimation and culture process of the microbial membrane further comprises a monitoring method for the primary acclimation and culture process of the microbial membrane, which comprises the following steps of:
(2-1) conveying a blank water sample to the plug-flow reactor, and detecting a water sample flowing out through a detection system, so as to obtain an initial signal;
(2-2) adding a standard solution of a pollutant with a known concentration into the blank water sample, conveying the water sample containing the standard solution of the pollutant with the known concentration to the plug-flow reactor, and detecting a water sample flowing out through a detection system, so as to obtain a water sample signal, wherein a difference between the water sample signal and the initial signal is taken as a response signal of the water sample; and
(2-3) judging a growth status of the microbial membrane by a change of the response signal, and obtaining the microbial membrane reactor when it is measured that the response signal of the water sample does not change any more.

3. The plug-flow microbial membrane reactor according to claim 1,
wherein the biodegradable organic matter comprises at least one of biodegradable saccharide, amino acid, aliphatic hydrocarbon, an organic acid and an alcohol organic matter.

4. The plug-flow microbial membrane reactor according to claim 1, wherein a five-day biochemical oxygen demand concentration of the biodegradable organic matter is 2 mg/L to 500 mg/L, an ammonia nitrogen concentration of the inorganic ammonium salt is 0.5 mg/L to 50 mg/L according to N, a mass concentration of the inorganic carbonate is 0.004 g/L to 0.4 g/L, and a nitrite nitrogen concentration of the inorganic nitrite is 0.5 mg/L to 5 mg/L according to N.

5. The plug-flow microbial membrane reactor according to claim 2, wherein the detection system comprises at least one of a dissolved oxygen analysis system, a total organic carbon analysis system, a chemical oxygen demand analysis system and a pH analysis system.

6. The plug-flow microbial membrane reactor according to claim 1, wherein the environmental water sample comprises at least one of a natural water body and waste water.

7. A detection system of a biodegradation reactor, comprising the plug-flow microbial membrane reactor according to claim 1, a power system, a detection system and a flow system for storing and conveying a fluid; and
one side of the plug-flow microbial membrane reactor is connected with a pipeline and the power system to form a closed system, a fluid in the flow system is conveyed to the plug-flow microbial membrane reactor through the pipeline and the power system, the fluid passes through the plug-flow microbial membrane reactor and then flows out from another side, and a fluid flowing out is detected through the detection system.

8. A detection method of a detection system of a biodegradation reactor by using the detection system of the biodegradation reactor according to claim 7, comprising the following steps of:
(1) taking a blank water sample as a fluid, and detecting the blank water sample by using the detection system of the biodegradation reactor, so as to obtain an initial signal; and
(2) detecting a signal of a water sample to be detected through the detection system of the biodegradation reactor, so as to obtain the signal of the water sample to be detected, wherein a difference between the signal of the water sample to be detected and the initial signal is taken as a response signal of the water sample to be detected.

9. The detection method of the detection system of the biodegradation reactor according to claim 8, comprising the following steps of:
(1) drawing a standard curve: taking standard samples containing pollutants with different concentrations as standard solutions, detecting the standard solutions by using the detection system of the biodegradation reactor, so as to obtain response signals of different standard solutions, and taking the concentrations of the standard solutions as abscissas and taking the response signals of the standard solutions as ordinates to draw the standard curve; and
(2) detecting the water sample to be detected: detecting the water sample to be detected by using the detection system of the biodegradation reactor, so as to obtain the response signal of the water sample to be detected, and calculating a detection result of the water sample to be detected through the standard curve.

10. The detection method of the detection system of the biodegradation reactor according to claim 8, wherein when used for detecting a biochemical oxygen demand of water quality, the acclimating agent is a biodegradable organic matter, and the detection system is a dissolved oxygen analysis system, a total organic carbon analysis system or a chemical oxygen demand analysis system.

11. The plug-flow microbial membrane reactor according to claim 1, wherein the inorganic ammonium salt comprises an inorganic matter containing ammonium ions.

12. The plug-flow microbial membrane reactor according to claim 1, wherein the inorganic carbonate comprises an inorganic matter containing carbonate ions.

13. The plug-flow microbial membrane reactor according to claim 1, wherein the inorganic nitrite comprises an inorganic matter containing nitrite ions.

14. The plug-flow microbial membrane reactor according to claim 1, wherein the construction method further comprises carrying out a secondary acclimation and culture process of the microbial membrane for the microbial membrane reactor, which specifically comprises the following steps of:
adding an acclimating agent into a water sample in a constructed microbial membrane reactor, and then conveying the water sample containing the acclimating agent to the plug-flow reactor for carrying out the secondary acclimation and culture process of the microbial membrane, so as to obtain the microbial membrane reactor, wherein the acclimating agent used in the secondary acclimation and culture process of the microbial membrane is different from the acclimating agent used in the primary acclimation and culture process of the microbial membrane.

15. The plug-flow microbial membrane reactor according to claim 2, wherein the blank water sample comprises a tap water sample or an unpolluted water sample.

16. The plug-flow microbial membrane reactor according to claim 1, wherein the plug-flow reactor is made of a material comprising at least one of polyurethane, polylactic acid and glass.

17. The plug-flow microbial membrane reactor according to claim 1, wherein a flow rate of the environmental water sample is 0.5 mL/min to 5 mL/min.

18. The plug-flow microbial membrane reactor according to claim 1, wherein a temperature of the constant-temperature water bath is 25° C. to 37° C.

19. The detection method of the detection system of the biodegradation reactor according to claim 8, wherein when used for detecting an ammonia nitrogen concentration of water quality, the acclimating agent is an inorganic ammonium salt, inorganic carbonate and/or inorganic nitrite, and the detection system is a pH analysis system or a dissolved oxygen analysis system.

20. The detection method of the detection system of the biodegradation reactor according to claim 8, wherein when used for synchronously detecting the biochemical oxygen demand and the ammonia nitrogen of water quality, the acclimating agent is a biodegradable organic matter, an inorganic ammonium salt, inorganic carbonate and/or inorganic nitrite, and the detection system is a dissolved oxygen analysis system.
